(19) United States
(12) Patent Application Publication
(10) Pub. No.: US 2005/0115568 A1
(43) Pub. Date: Jun. 2, 2005

(54) Prophylactic device

(76) Inventor: Kathleen K. Martin, Laytonville, CA (US)

Correspondence Address: FOLEY AND LARDNER;SUITE 500, 3000 K STREET NW, WASHINGTON, DC, 20007, US

(21) Appl. No.: 10/724,864

(22) Filed: Dec. 2, 2003

(51) Int. Cl. A61F 6/04

(52) U.S. Cl. 128/844

(57) ABSTRACT

A prophylactic device includes a panty having a crotch portion and an elongated pouch disposed on the crotch portion. The pouch is made of an elastic material, and the panty and the pouch are integrally formed. The pouch can include longitudinal pleats, and an interior of the panty can include a plurality of raised protrusions positioned to facilitate positioning of the panty on a user. The rear portion of the panty can include a sculpted portion that is configured to curve inward to conform to the shape of a user's lower back when the panty is in an in-use configuration. The prophylactic device can be produced by a dipping process that results in a device with a thicker pouch than panty.

### BACKGROUND OF THE INVENTION

The present invention relates to a prophylactic and contraceptive device.

Condoms used during sexual intercourse for contraception and prophylaxis, including preventing the spread of sexually transmitted diseases (STDs) and auto immune deficiency syndrome (AIDS), traditionally are worn by males. Male condoms typically are tube-like pouches made of liquid impermeable material, which roll onto and tightly conform to the penis in order to contain seminal and other bodily fluids released by the male.

However, even with proper condom usage, it is still possible for partners to exchange bodily fluids during sexual activity. When such fluid contacts vaginal and other bodily parts, transmission of STDs and AIDS may occur.

Moreover, with traditional male condoms, a woman lacks the ability to control contraception and prophylaxis. Instead, she must persuade her male partner to voluntarily wear a condom. Donning a condom takes time and may detract from the romantic mood. Additionally, use of a condom may diminish sensitivity and sexual pleasure. Thus, convincing a male partner to wear a condom can be difficult. In situations involving rape, it is highly unlikely that the woman will have a condom on hand and even more unlikely that she will be able to convince the rapist to wear the condom.

Although female condoms exist, they are expensive, difficult to use, and occasionally cause pain to the woman during intercourse. Even if such devices could be used with relative ease, their complex construction and resulting high cost makes them unsuited to countries with depressed economies, with a high incidence of AIDS, and/or with a high incidence of rape.

### SUMMARY OF THE INVENTION

An aspect of the present invention relates to a prophylactic device. The prophylactic device includes a panty having a crotch portion and an elongated pouch disposed on the crotch portion. The pouch is made of an elastic material, and the panty and the pouch are integrally formed.

Another aspect of the present invention relates to a prophylactic device that includes a panty having a crotch portion and an elongated pouch disposed on the crotch portion, wherein the pouch includes longitudinal pleats.

Another aspect of the present invention relates to a prophylactic device that includes a panty having a crotch portion and an elongated pouch disposed on the crotch portion, wherein an interior of the panty includes a plurality of raised protrusions to facilitate positioning of the panty on a user.

Another aspect of the present invention relates to a prophylactic device that includes a panty having a crotch portion and an elongated pouch disposed on the crotch portion, wherein a rear portion of the panty includes a sculpted portion configured to curve inward to conform to a shape of a user's lower back when the panty is in an in-use configuration.

Another aspect of the present invention relates to a prophylactic device that includes a panty having a crotch portion and an elongated pouch disposed on the crotch portion, wherein the panty is thicker than the pouch.

Yet another aspect of the present invention relates to a method for manufacturing a prophylactic device. The method includes providing a mold form that has a panty casting portion and a pouch casting portion. The method includes dipping the mold form into a first coagulant until the pouch casting portion and the panty casting portion are submerged in the first coagulant, dipping the form into a second coagulant until the pouch casting portion is submerged in the second coagulant, dipping the form into latex a first time until the pouch casting portion and the panty casting portion are submerged in the latex, dipping the form into the latex a second time until the pouch casting portion is submerged in the latex, and removing the casting from the form.

It is to be understood that both the foregoing general description and the following detailed description are exemplary and explanatory only and are not restrictive of the invention claimed.

### BRIEF DESCRIPTION OF THE DRAWINGS

The accompanying drawings, which are incorporated in and constitute a part of this specification, illustrate preferred embodiments of the invention and together with the description, serve to explain principles of the invention.

FIG. 1 is a perspective view of an embodiment of a prophylactic device according to the present invention showing a panty and a pouch.

FIG. 2 is a front perspective view of the embodiment of FIG. 1 showing the panty on a female user and the pouch inserted into a vaginal cavity of the user.

FIG. 3 is a side view of the embodiment of FIG. 1.

FIG. 4 is a side view of the embodiment of FIG. 1 showing the pouch in a folded configuration.

FIG. 5 is a perspective view of another embodiment of a prophylactic device according to the present invention.

FIG. 6 is a schematic view showing a pattern of raised protrusions according to an embodiment of the present invention.

FIG. 7 is a side elevation view of a mold form according to an embodiment of the present invention.

FIG. 8 is a front elevation view of a mold form according to an embodiment of the present invention.

FIG. 9 is a top plan view of a mold form according to an embodiment of the present invention.

FIG. 10 is a flow chart illustrating a casting process according to an embodiment of the present invention.

### DETAILED DESCRIPTION

Reference will now be made in detail to presently preferred embodiments of the invention, examples of which are illustrated in the accompanying drawings. An effort has been made to use the same reference numbers throughout the drawings to refer to the same or like parts.

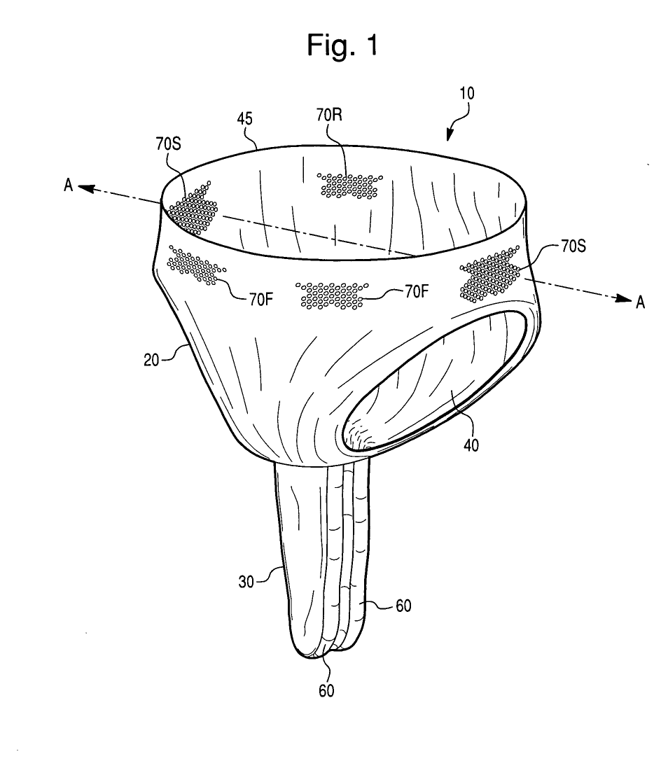

FIGS. 1-3 show an embodiment of a prophylactic device 10 suitable for use by a female. The prophylactic device 10 includes a panty 20 and a pouch 30.

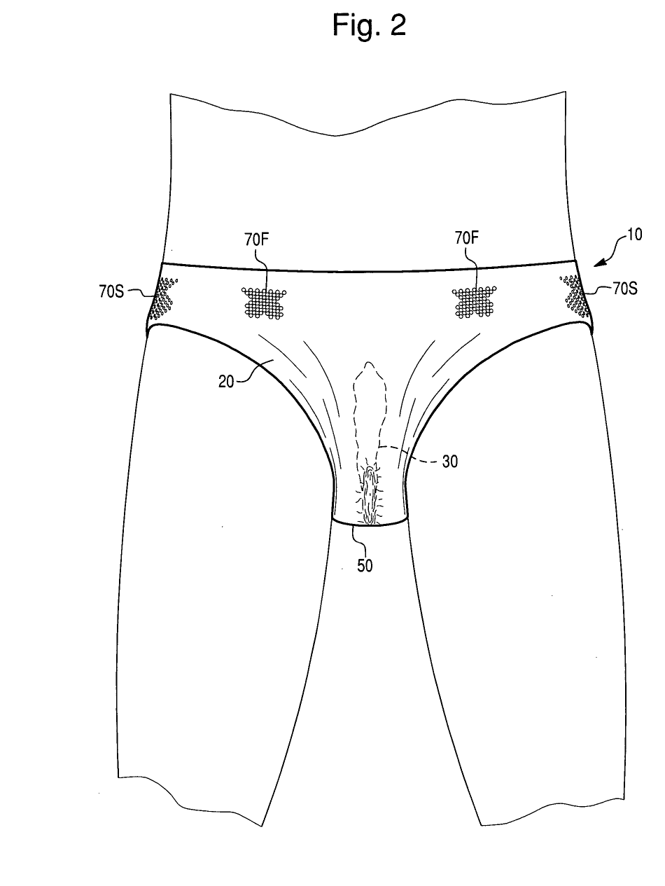

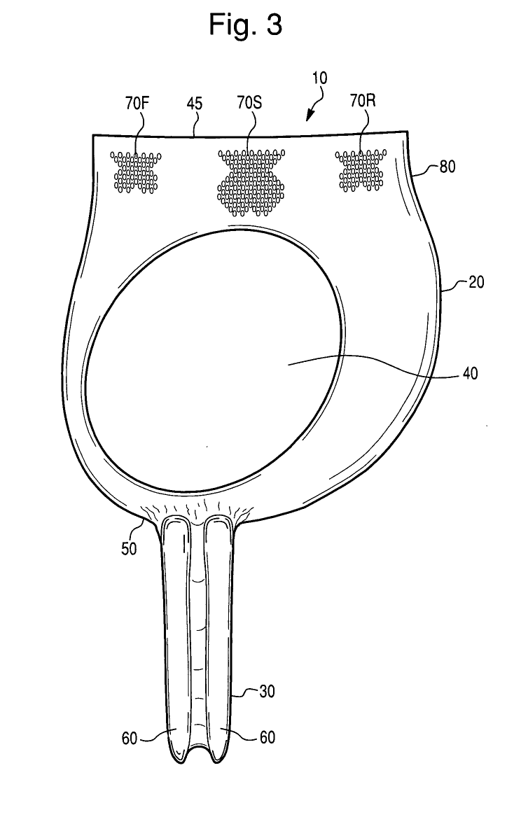

The panty 20 has a shape similar to that of conventional underpants, as shown in FIG. 1. The panty 20 includes leg apertures 40, as shown in FIGS. 1 and 3, so that the panty 20 can be worn by a female user, as shown in FIG. 2. The panty 20 has a crotch portion 50 disposed between the leg apertures 40, as shown best in FIG. 2. The panty 20 additionally includes a waistband 45 disposed at a top portion of the panty 20 along a circumference of the panty 20, as indicated in FIGS. 1 and 3. The panty 20 is configured so that the panty 20 substantially covers the buttocks and genital area of the user.

The pouch 30 has an elongated shape and is disposed on the crotch portion 50 of the panty 20. The pouch 30 is configured to move from an open configuration, in which the pouch 30 extends outwardly from the panty 20 as shown in FIG. 1, to an inverted configuration, in which the pouch 30 extends inwardly toward an interior portion of the panty as shown in FIG. 2. When the prophylactic device 10 is worn by a female user, the pouch 30 is substantially aligned with the vaginal opening of the user. The pouch 30 can be moved into the inverted configuration and inserted into the vaginal cavity of the user. In the inserted configuration, the pouch 30 is adapted to receive the penis of a male partner during sexual intercourse between the female user and the male partner. The pouch 30 is configured to perform a barrier function so that bodily fluids are not exchanged between the female user and the male partner.

According to an embodiment of the invention, the panty 20 and the pouch 30 are integrally formed so that the prophylactic device 10 comprises a single unitary body. In this regard, the prophylactic device 10 is seamless. In addition, the prophylactic device 10 is made of an elastic material that is impermeable to fluids. For example, the prophylactic device 10 may be made of latex; a polymer material, such as polyurethane; or a combination of latex and polymer material. Because the prophylactic device 10 is made of an elastic material, the prophylactic device can conform to fit any sexually mature user.

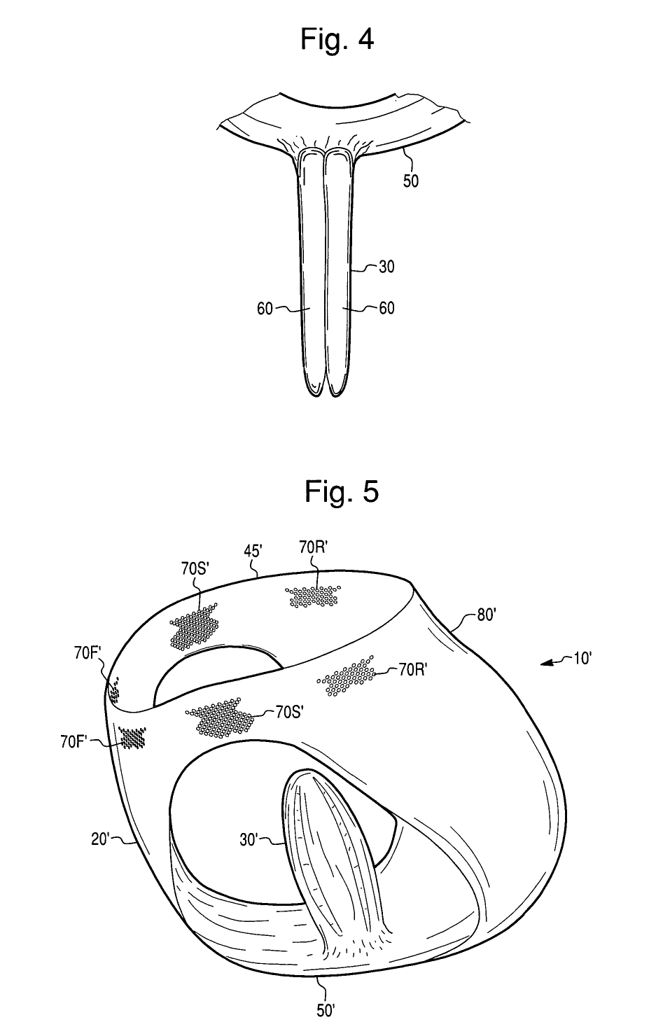

The pouch 30 can include longitudinal pleats 60 disposed on the sides of the pouch 30. For example, as shown in FIGS. 1 and 3, the pouch 30 can include two longitudinal pleats 60 on each side of the pouch 30 for a total of four longitudinal pleats 60. It will be understood, however, that the pouch 30 can include fewer pleats or more pleats than shown in FIGS. 1 and 3. These longitudinal pleats 60 are generally parallel to a longitudinal axis of the elongated pouch 30. The longitudinal pleats 60 are configured to fold lengthwise along the pouch, as shown in FIG. 4. The longitudinal pleats 60 enable the pouch 30 to move from a relatively flat, compact, folded configuration (shown in FIG. 4) to an open configuration (shown in FIGS. 1 and 3) and from an open configuration to an expanded configuration (shown in FIG. 5). In the expanded configuration, a circumference of the pouch 30 is larger than a circumference of an average adult penis, which reduces the likelihood that the pouch 30 will pull out of a vaginal or anal cavity during use. Thus, the pouch 30 is large enough to line the inside of a vaginal or anal cavity and loose enough to prevent the pouch from riding the penis out of the cavity. Additionally, because the pleats 60 fold inward, the size of the pouch 30 appears small, which improves the visual appearance and overall acceptability of the prophylactic device 10. In this regard, when the longitudinal pleats 60 are in the folded configuration, the user can comfortably wear the prophylactic device 10 with the pouch 30 tucked unobtrusively next to the panty 20. Also, the panty 20 can be folded along its front-to-back midline in a flattened configuration, which, with the folded pouch 30, creates a compact arrangement for product packaging.

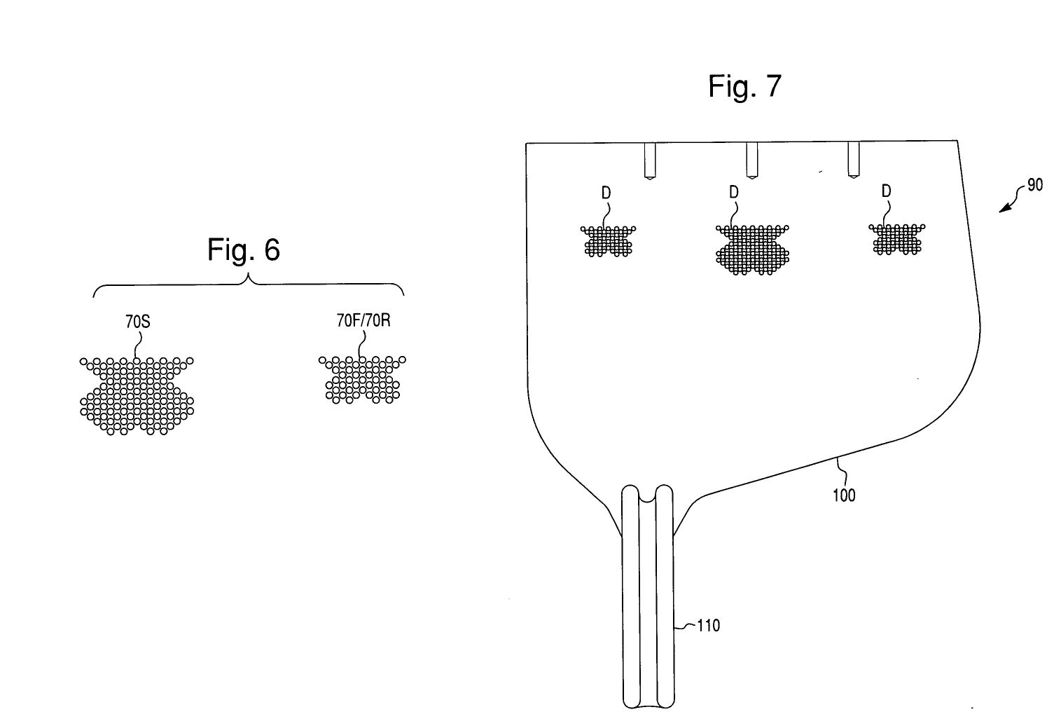

An upper portion of the panty 20 can include a plurality of raised protrusions 70S, 70F, 70R. The raised protrusions 70S, 70F, 70R are disposed closer to the waistband 45 of the prophylactic device 10 than to the crotch portion 50. The raised protrusions 70S, 70F, 70R can be grouped into patterns, as shown, for example, in FIG. 6. In one embodiment, the panty 20 can include six patterns of raised protrusions 70S, 70F, 70R located so as to assist the user in unrolling the waistband 45 or in adjusting the prophylactic device 10 for optimum fit and maximum comfort. For example, as shown in FIGS. 1-3 and 5, a first pattern of raised protrusions 70S may be located along the panty's transverse axis A-A on a left side of the panty 20, and a second pattern of raised protrusions 70S may be located along the transverse axis A-A on a right side of the panty 20. The panty 20 also can include patterns of raised protrusions 70F on a front side of the panty forward of the transverse axis A-A, a first pattern 70F being equidistant between a midpoint of the front portion of the panty 20 and a point at which the transverse axis A-A intersects the left side of the panty 20, and a second pattern 70F being equidistant between a midpoint of the front portion of the panty 20 and a point at which the transverse axis A-A intersects the right side of the panty 20. The panty 20 further can include patterns of raised protrusions 70R on a rear side of the panty rearward of the transverse axis A-A, a first pattern 70R being equidistant between a midpoint of the rear portion of the panty 20 and a point at which the transverse axis A-A intersects the left side of the panty 20, and a second pattern 70R being equidistant between a midpoint of the rear portion of the panty 20 and a point at which the transverse axis A-A intersects the right side of the panty 20. It will be understood that other embodiments of the prophylactic device 10 can include more than six, or less than six, patterns of raised protrusions.

The raised protrusions 70S, 70F, 70R project inwardly toward an interior of the panty 20 and result in corresponding recesses located on an exterior portion of the panty 20. The raised protrusions 70S, 70F, 70R are preferably rounded and are small enough to avoid causing discomfort to a user. For example, the raised protrusions 70S, 70F, 70R may have a diameter of approximately 1/8 inch and a height of approximately 1/16 inch.

The shape of the panty 20 can be configured so that when the panty 20 is moved into an in-use configuration (that is, a configuration where a user is wearing the panty 20), a sculpted portion 80 forms along a center rear portion of the waistband 45. That is, as shown in FIG. 3, the rear portion of the panty 20 can include a rounded buttock portion, visible in the in-use configuration, and the waistband 45 disposed above the buttock portion at the top portion of the panty 20; the sculpted portion is disposed on the waistband 45. The sculpted portion 80 (shown in FIGS. 3 and 5) is configured to curve inward to conform to the shape of a user's back, which improves the fit of the panty 20 and increases user comfort. The sculpted portion 80 eliminates excess fabric from the panty 20, thereby enabling the panty 20 to conform to the shape of a user's body and preventing the panty 20 from presenting a baggy and visually unacceptable appearance when the panty 20 is worn by the user.

The panty 20 can be thicker than the pouch 30 to promote comfort and ease of use. A thin pouch 30 is unobtrusive and enables the user and the male partner to experience greater sexual sensitivity than with a thick pouch. A thick panty 20 provides a heavier form than the thin pouch 30 and enables the user to easily manipulate and comfortably wear the panty 20. For example, a thickness of the panty 20 may be 14 to 16 mil, and a thickness of the pouch may be 8 to 10 mil. The pouch 30 may include two layers of material, such as latex.

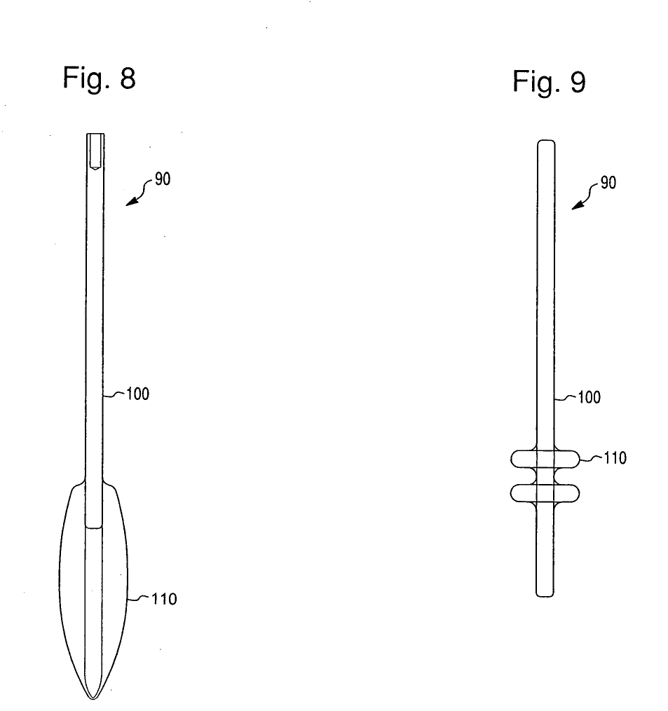

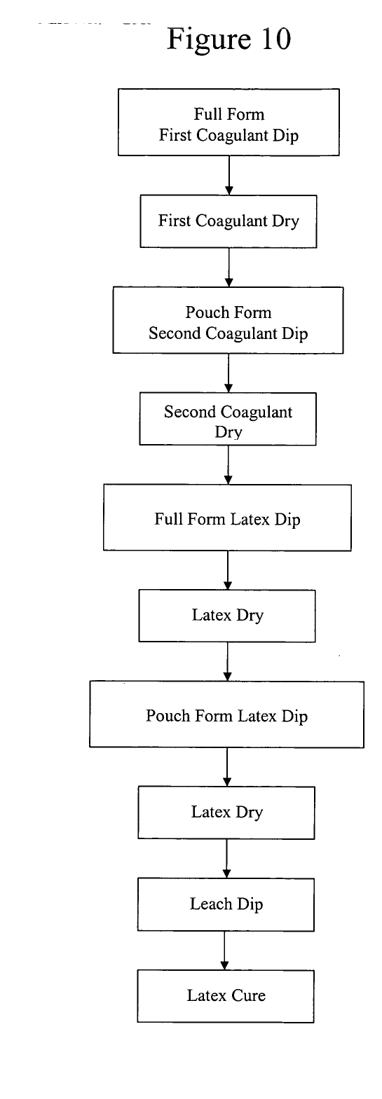

A method of producing a prophylactic device according to the invention now will be described in connection with FIGS. 6-10. The prophylactic device 10 is preferably formed as a casting. The casting (or molding) process includes providing a mold form 90 (shown in FIGS. 7-9) for forming a shape of the casting. The form 90 includes a portion 100 configured to produce a panty casting and a portion 110 configured to produce a pouch casting. The casting process is described below, and an illustrative example of an embodiment of the process is provided in FIG. 10.

The form 90 first is dipped into a first coagulant until the portions 100 and 110 are submerged in the coagulant. The first coagulant may be, for example, heavy wall coagulant. The first coagulant is formulated so that a casting material (such as latex) will adhere to the first coagulant to form a heavy (or thick) wall. The temperature of the first coagulant is, for example, 135 degrees Fahrenheit. The form 90 is removed from the first coagulant and is subjected to a drying process for, for example, about 2 minutes at 212 degrees Fahrenheit. The form 90 then is dipped into a second coagulant until only the portion 110 is submerged. The second coagulant forms a coating over the first coagulant on the portion 110. The second coagulant may be, for example, light wall coagulant. The second coagulant is formulated so that a casting material (such as latex) will adhere to the second coagulant to form a light (or thin) wall. The temperature of the second coagulant is, for example, 130 degrees Fahrenheit. The form 90 is removed from the second coagulant and is subjected to a drying process for, for example, two minutes at 212 degrees Fahrenheit. The form 90 is next dipped into latex a first time until the portions 100 and 110 are submerged in the latex. The temperature of the latex is, for example, at ambient temperature (that is, at about 72 degrees Fahrenheit). The form 90 is removed from the latex and is subjected to a drying process for, for example, two minutes at 212 degrees Fahrenheit. The form 90 is dipped into the latex a second time until only the portion 110 is submerged, and, again, the latex is at ambient temperature. Portion 110 is dipped into the latex a second time in accordance with industry standards for condom manufacture. The second latex dip improves the reliability of the casting by preventing pinholes from forming in the pouch portion of the casting. The form 90 is removed from the latex and is subjected to a drying process for, for example, two minutes at 212 degrees Fahrenheit.

After the coagulant and latex dips are complete, the form 90 is subjected to a leaching process. The leaching process (or leach dip) removes impurities from the latex. For example, the leaching process may include dipping the form 90 into hot water. This leach dip can last, for example, for twenty minutes at 130 degrees Fahrenheit. The form 90 next undergoes a curing process for 30 minutes at 212 degree Fahrenheit, for example, and then a cool down. The latex cure, which sets the latex, may be performed, for example, by placing the form 90 into a high temperature oven. Prior to removing the cooled latex casting from the form 90, powder is applied to a portion of the casting to assist in removing the casting from the form 90. For example, power can be sprinkled on an exterior portion of the casting at a top of the form 90 to permit detachment of the casting from the form. Additional power can be added to the casting as needed (for example, to an interior portion or an exterior portion of the casting) as the casting is removed from the form 90. The completed casting can be removed from the form 90 using any conventional removal methods. For example, the casing can be removed manually by peeling the completed casting from the form 90 or by air stripping. A casting having a one-layer panty portion and a two-layer pouch portion can be produced by this casting process.

The form 90 can include a plurality of depressions D disposed on the portion of the form 90 configured to produce a panty casting. The depressions D are configured to receive a flow of latex when the form is dipped so that a plurality of raised protrusions are formed on the panty casting. For example, the depressions D may include spherical indentations having a diameter of approximately 1/8 inch and a depth of approximately 1/16 inch.

Another embodiment of a prophylactic device 10′ is shown in FIG. 5. This prophylactic device 10′ is similar to prophylactic device 10 of FIGS. 1-3; however, as can be seen in FIG. 5, the pouch 30′ of prophylactic device 10′ is located more rearwardly on the crotch portion 50′. When the prophylactic device 10′ is worn by a user, the pouch 30′ is substantially aligned with an anal opening of the user. The pouch 30′ can be moved into the inverted configuration and inserted into the anal cavity of the user. In the inserted configuration, the pouch 30′ is adapted to receive the penis of a male partner during intercourse between the user and the male partner. The pouch 30′ is configured to perform a barrier function so that bodily fluids are not exchanged between the user and the male partner. According to this embodiment, the panty 20′ may optionally include an aperture (not shown) on the front portion of the panty 20′ to accommodate the penis of a male user.

Other embodiments of the invention will be apparent to those skilled in the art from consideration of the specification and practice of the invention disclosed herein. It is intended that the specification and examples be considered as exemplary only.

1. A prophylactic device, comprising:
a panty including a crotch portion; and
an elongated pouch disposed on the crotch portion,
wherein the panty and the pouch are integrally formed, and
wherein the pouch is formed of an elastic material.

2. The prophylactic device of claim 1, wherein the pouch is configured to move from an open configuration to an inverted configuration.

3. The prophylactic device of claim 2, wherein a portion of the pouch is configured to be disposed within a vaginal cavity of a user when the pouch is in the inverted configuration.

4. The prophylactic device of claim 2, wherein a portion of the pouch is configured to be disposed within an anal cavity of a user when the pouch is in the inverted configuration.

5. The prophylactic device of claim 1, wherein the prophylactic device is comprised of latex.

6. The prophylactic device of claim 1, wherein the prophylactic device is comprised of a polymer material.

7. The prophylactic device of claim 1, wherein the panty is configured to substantially cover a user's buttocks and genital area.

8. The prophylactic device of claim 1, wherein a thickness of the panty is greater than a thickness of the pouch.

9. The prophylactic device of claim 1, wherein an interior of the panty includes a plurality of raised protrusions positioned to facilitate positioning of the panty on a user.

10. The prophylactic device of claim 1, wherein the prophylactic device includes a rear portion including a sculpted portion, and wherein the sculpted portion is configured to curve inward to conform to a shape of a user's lower back when the panty is in an in-use configuration.

11. The prophylactic device of claim 1, wherein the pouch can expand from a folded configuration to an open configuration, and wherein the pouch includes longitudinal pleats.

12. The prophylactic device of claim 1, further comprising an aperture disposed on the crotch portion, wherein the aperture is configured to accommodate a penis of a user.

13. A prophylactic device, comprising:
a panty including a crotch portion; and
an elongated pouch disposed on the crotch portion,
wherein the pouch includes longitudinal pleats.

14. The prophylactic device of claim 13, wherein the pouch includes at least four longitudinal pleats.

15. The prophylactic device of claim 13, wherein the longitudinal pleats are configured to fold lengthwise along the pouch.

16. The prophylactic device of claim 13, wherein the pouch can expand from a folded configuration to an open configuration.

17. The prophylactic device of claim 13, wherein the pouch can expand from an open configuration to an expanded configuration.

18. The prophylactic device of claim 13, wherein the pouch is configured to move from an open configuration to an inverted configuration.

19. The prophylactic device of claim 18, wherein the pouch in the open configuration extends outward from the crotch portion, and wherein the pouch in the inverted configuration extends inward from the crotch portion.

20. The prophylactic device of claim 18, wherein a portion of the pouch is configured to be disposed within a vaginal cavity of a user when the pouch is in the inverted configuration.

21. The prophylactic device of claim 18, wherein a portion of the pouch is configured to be disposed within an anal cavity of a user when the pouch is in the inverted configuration.

22. The prophylactic device of claim 13, wherein the panty and the pouch are integrally formed.

23. The prophylactic device of claim 13, wherein the panty is configured to substantially cover a user's buttocks and genital area.

24. The prophylactic device of claim 13, wherein a thickness of the panty is greater than a thickness of the pouch.

25. The prophylactic device of claim 13, wherein the prophylactic device is comprised of latex.

26. The prophylactic device of claim 13, wherein the prophylactic device is comprised of a polymer material.

27. The prophylactic device of claim 13, wherein an interior of the panty includes a plurality of raised protrusions positioned to facilitate positioning of the panty on a user.

28. The prophylactic device of claim 13, wherein the prophylactic device includes a rear portion including a sculpted portion, and wherein the sculpted portion is configured to curve inward to conform to a shape of a user's lower back when the panty is in an in-use configuration.

29. A prophylactic device, comprising:
a panty including a crotch portion; and
an elongated pouch disposed on the crotch portion,
wherein an interior of the panty includes a plurality of raised protrusions positioned to facilitate positioning of the panty on a user.

30. The prophylactic device of claim 29, wherein the raised protrusions are rounded.

31. The prophylactic device of claim 29, wherein the raised protrusions are grouped to form a pattern.

32. The prophylactic device of claim 29, wherein the raised protrusions are disposed closer to a waistband of the panty than to the crotch portion of the panty.

33. The prophylactic device of claim 29, wherein the panty includes a transverse axis, and wherein the panty includes a first pattern of raised protrusions disposed on a first side of the panty along the transverse axis and a second pattern of raised protrusions disposed on a second side of the panty along the transverse axis.

34. The prophylactic device of claim 29, wherein the panty includes a transverse axis, and wherein the panty includes first and second patterns of raised protrusions disposed on a front side of the panty forward of the transverse axis.

35. The prophylactic device of claim 34, wherein the first pattern of raised protrusions is disposed equidistant from a midpoint of the front portion and a point at which the transverse axis intersects a first side of the panty, and wherein the second pattern of raised protrusions is disposed equidistant from the midpoint of the front portion and a point at which the transverse axis intersects a second side of the panty.

36. The prophylactic device of claim 29, wherein the panty includes a transverse axis, and wherein the panty includes first and second patterns of raised protrusions disposed on a rear side of the panty rearward of the transverse axis.

37. The prophylactic device of claim 36, wherein the first pattern of raised protrusions is disposed equidistant from a midpoint of the rear portion and a point at which the transverse axis intersects a first side of the panty, and wherein the second pattern of raised protrusions is disposed equidistant from the midpoint of the rear portion and a point at which the transverse axis intersects a second side of the panty.

38. The prophylactic device of claim 29, wherein the panty and the pouch are integrally formed.

39. The prophylactic device of claim 29, wherein the panty is configured to substantially cover a user's buttocks and genital area.

40. The prophylactic device of claim 29, wherein a thickness of the panty is greater than a thickness of the pouch.

41. The prophylactic device of claim 29, wherein the prophylactic device is comprised of latex.

42. The prophylactic device of claim 29, wherein the prophylactic device is comprised of a polymer material.

43. The prophylactic device of claim 29, wherein the prophylactic device includes a rear portion including a sculpted portion, and wherein the sculpted portion is configured to curve inward to conform to a shape of a user's lower back when the panty is in an in-use configuration.

44. A prophylactic device, comprising:
a panty including a crotch portion; and
an elongated pouch disposed on the crotch portion,
wherein a rear portion of the panty includes a sculpted portion, and wherein the sculpted portion is configured to curve inward to conform to a shape of a user's lower back when the panty is in an in-use configuration.

45. The prophylactic device of claim 44, wherein the rear portion includes a buttock portion and a waist portion disposed above the buttock portion, and wherein the sculpted portion is disposed on the waist portion.

46. The prophylactic device of claim 44, wherein the panty and the pouch are integrally formed.

47. The prophylactic device of claim 44, wherein the panty is configured to substantially cover a user's buttocks and genital area.

48. The prophylactic device of claim 44, wherein a thickness of the panty is greater than a thickness of the pouch.

49. The prophylactic device of claim 44, wherein the prophylactic device is comprised of latex.

50. The prophylactic device of claim 44, wherein the prophylactic device is comprised of a polymer material.

51. A method of producing a prophylactic device, comprising:
providing a form for a casting including a portion configured to produce a panty casting and a portion configured to produce a pouch casting, wherein the panty casting includes a crotch portion, and wherein the pouch casting is disposed on the crotch portion of the panty casting;
dipping the form into a first coagulant until the pouch casting portion and the panty casting portion are submerged in the first coagulant;
dipping the form into a second coagulant until the pouch casting portion is submerged in the second coagulant;
dipping the form into latex a first time until the pouch casting portion and the panty casting portion are submerged in the latex;
dipping the form into the latex a second time until the pouch casting portion is submerged in the latex; and
removing the casting from the form.

52. The method of claim 51, further comprising, after the step of dipping the form into a first coagulant, removing the form from the first coagulant and subjecting the form to a drying process.

53. The method of claim 51, further comprising, after the step of dipping the form into a second coagulant, removing the form from the second coagulant and subjecting the form to a drying process.

54. The method of claim 51, further comprising, after the step of dipping the form into latex a first time, removing the form from the latex and subjecting the form to a drying process.

55. The method of claim 51, further comprising, after the step of dipping the form into the latex a second time, removing the form from the coagulant and subjecting the form to a drying process.

56. The method of claim 51, further comprising subjecting the form to a leaching process.

57. The method of claim 51, further comprising subjecting the form to a curing process.

58. The method of claim 51, further comprising depositing powder on an exterior portion of the casting.

59. The method of claim 51, further comprising introducing powder into an interior portion of the casting.

60. The method of claim 51, wherein the first coagulant and the second coagulant comprise a liquid.

61. The method of claim 51, wherein the latex comprises a liquid.

62. The method of claim 51, wherein the first coagulant and the second coagulant are formulated to promote adherence of the latex.

63. The method of claim 56, wherein the leaching process includes dipping the form into water.

64. The method of claim 51, wherein the form includes a plurality of depressions disposed on the portion of the form configured to produce a panty casting, and wherein the depressions are configured to receive a flow of latex when the form is dipped so that a plurality of raised protrusions are formed on the panty casting.

65. A prophylactic device, comprising:
a panty including a crotch portion; and
an elongated pouch disposed on the crotch portion,
wherein the panty is thicker than the pouch.

66. The prophylactic device of claim 65, wherein the prophylactic device is comprised of latex.

67. The prophylactic device of claim 65, wherein the prophylactic device is comprised of a polymer material.

68. The prophylactic device of claim 65, wherein a thickness of the panty is approximately 14 to 16 mil.

69. The prophylactic device of claim 65, wherein the pouch comprises at least two layers of latex.

70. The prophylactic device of claim 65, wherein a thickness of the pouch is approximately 8 to 10 mil.

71. The prophylactic device of claim 65, wherein the panty and the pouch are integrally formed.

72. The prophylactic device of claim 65, wherein the panty is configured to substantially cover a user's buttocks and genital area.
